SEQUENCE LISTING (12 sequences)

SEQ ID NO: 1 - RNA, 30 nt, synthetic construct
  source 1..30: mol_type = other RNA
gtgtatagatgggaaagggttctatacgca

SEQ ID NO: 2 - RNA, 11 nt, synthetic construct
  source 1..11: mol_type = other RNA
tgggaaagggt

SEQ ID NO: 3 - RNA, 56 nt, synthetic construct
  source 1..56: mol_type = other RNA
ggcgcactggcgctgcgcctgggaaagggtggcgccaatcgtagcgtgtcggcgcc

SEQ ID NO: 4 - RNA, 18 nt, synthetic construct
  source 1..18: mol_type = other RNA
ggggcgggtggggagggg

SEQ ID NO: 5 - DNA, 11 bp, synthetic construct
  source 1..11: mol_type = other DNA
tgggaaagggt

SEQ ID NO: 6 - RNA, 31 nt, synthetic construct
  source 1..31: mol_type = other RNA
gtaaaatagatgggaaagggttctatacgca

SEQ ID NO: 7 - RNA, 48 nt, synthetic construct
  source 1..48: mol_type = other RNA
ggcgccaatcgtagcgtgtcggtgggaaagggtcactggcgctgcgcc

SEQ ID NO: 8 - RNA, 50 nt, synthetic construct
  source 1..50: mol_type = other RNA
ggcgccaatcgtagcgtgtcggctgggaaagggtgcactggcgctgcgcc

SEQ ID NO: 9 - RNA, 52 nt, synthetic construct
  source 1..52: mol_type = other RNA
ggcgccaatcgtagcgtgtcggcgtgggaaagggtcgcactggcgctgcgcc

SEQ ID NO: 10 - RNA, 50 nt, synthetic construct
  source 1..50: mol_type = other RNA
ggcgcactggcgctgctgggaaagggtgccaatcgtagcgtgtcggcgcc

SEQ ID NO: 11 - RNA, 52 nt, synthetic construct
  source 1..52: mol_type = other RNA
ggcgcactggcgctgcgtgggaaagggtcgccaatcgtagcgtgtcggcgcc

SEQ ID NO: 12 - RNA, 54 nt, synthetic construct
  source 1..54: mol_type = other RNA
ggcgcactggcgctgcgctgggaaagggtgcgccaatcgtagcgtgtcggcgcc

(19) United States
(12) Patent Application Publication
(10) Pub. No.: US 2025/0075212 A1
(43) Pub. Date: Mar. 6, 2025

(54) L-RNA APTAMERS, RECOMBINANT POLYNUCLEOTIDES, AND USES THEREOF IN TREATING ALZHEIMERS DISEASE

(71) Applicant: CITY UNIVERSITY OF HONG KONG, Kowloon (HK)

(72) Inventor: Chun Kit KWOK, Kowloon (HK)

(21) Appl. No.: 18/795,168

(22) Filed: Aug. 5, 2024

Related U.S. Application Data
(60) Provisional application No. 63/580,950, filed on Sep. 6, 2023.

(51) Int. Cl. C12N 15/113; C12P 19/34

(52) U.S. Cl. CPC C12N 15/113; C12P 19/34; C12N 2310/11; C12N 2310/531

(57) ABSTRACT

Disclosed herein is an L-RNA aptamer specific to 3′-untranslated region (3′-UTR) of amyloid precursor protein (APP) messenger RNA (mRNA). According to some embodiments of the present disclosure, the L-RNA comprises the nucleotide sequence of SEQ ID NO: 1. Also disclosed herein are a recombinant polynucleotide derived from the L-RNA aptamer, and uses of the L-RNA aptamer in treating Alzheimer's disease (AD).

### CROSS-REFERENCE TO RELATED APPLICATIONS

This application relates to and claims the benefits of U.S. Provisional Application No. 63/580,950 filed Sep. 6, 2023; the content of the application is incorporated herein by reference in the entirety.

### SEQUENCE LISTING XML

The present application is being filed along with a Sequence Listing XML in electronic format. The Sequence Listing XML is provided as an XML file entitled HP0317US_SEQ, created Jun. 27, 2024, which is 12 Kb in size. The information in the electronic format of the Sequence Listing XML is incorporated herein by reference in its entirety.

### BACKGROUND OF THE INVENTION

### 1. Field of the Invention

The present disclosure in general relates to the field of L-form ribonucleic acid (L-RNA) aptamers. More particularly, the present disclosure relates to a novel L-RNA aptamer, and its use in treating Alzheimer's disease (AD).

### 2. Description of Related Art

Guanine (G)-rich sequences in DNA and RNA can form into nucleic acid structure motifs referred to as G-quadruplex (G4) structures, which are assembled by two or more G-tetrads that are connected by loop nucleotides, and further stabilized by monovalent cations (K⁺>Na⁺>Li⁺). RNA G-quadruplexes (rG4s) are typically in parallel topology, and a number of studies suggested that they can be detected in different classes of RNA such as messenger RNAs (mRNAs), long non-coding RNAs (lncRNAs), microRNAs (miRNAs), transfer RNAs (tRNAs), and more. In addition, functional rG4s are reported in diverse organisms, including human, mouse, plants, bacteria, and others, highlighting their fundamental importance in biology across the tree of life. rG4s have been demonstrated to play roles in many biochemical processes, including regulation of transcription, RNA splicing, and translation, as well as control of RNA turnover and mRNA targeting. From transcriptome-wide and transcript-specific studies, a parallel rG4 at the 3′-untranslated region (3′-UTR) of APP mRNA was previously identified and characterized, and the analytic results revealed the inhibitory role of APP 3′UTR rG4 on APP translation. Amyloid precursor protein (APP) is an integral membrane protein that is commonly expressed in human tissues, and it is the precursor protein of amyloid β (Aβ) peptides. Studies have shown that the abnormal expression of APP is harmful, as such, developing targeting tools to control APP expression is of great significance.

Major G4 targeting tools include the G4 chemicals, such as pyridostatin, PhenDC3, and G4 antibody/nanobody such as BG4 and SG4. They have been used to detect G4 formation and control G4-linked functions both in vitro and in cells. While they have strong binding preference to G4 motifs over non-G4s, they are generally not sufficiently selective to distinguish between different G4s, raising an overarching question in the community that whether novel G4-targeting tools can be developed for precise G4 targeting. Aptamers are short single-stranded DNA or RNA oligonucleotides that fold into unique structure scaffold and bind to target of interest, including protein, cells, small molecules, and nucleic acid structures. L-Aptamers are composed of L-RNA (enantiomeric form of naturally occurring D-RNA) that is unnatural, so they cannot be recognized by nucleases and cannot not trigger immunogenicity, which greatly enhance their biostability in biological media. They are usually developed using in vitro selection, systematic evolution of ligands by exponential enrichment (SELEX). Recently, a robust SELEX platform (rG4-SELEX) was developed to generate L-RNA aptamers that target rG4, in which these aptamers can recognize its target and control gene regulation in cells by targeting rG4 of interest. However, their binding specificity are non-ideal due to their off-target binding to other similar rG4s.

In view of the foregoing, there is a continuing interest in developing a novel L-RNA aptamer that exhibits binding specificity towards rG4 target of interest (e.g., APP 3′UTR rG4) thereby efficiently detecting the expression of the rG4 target and/or treating the diseases associated with/caused by the rG4 target, e.g., Alzheimer's disease (AD).

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the present disclosure is directed to an L-RNA aptamer specific to the 3′-UTR of APP mRNA. According to some embodiments of the present disclosure, the L-RNA aptamer comprises the nucleotide sequence of “GUGUAUAGAUGGGAAAGGGUUCUAUACGCA” (SEQ ID NO: 1) that is characterized by having a loop region consisting of the nucleotide sequence of “UGGGAAAGGGU” (SEQ ID NO: 2).

According to certain embodiments of the present disclosure, the L-RNA aptamer is produced by a method comprising the steps of,
(a) providing a deoxyribonucleic acid (DNA) library comprising a first plurality of DNAs respectively having randomized sequences;
(b) producing a first plurality of D-form RNAs (D-RNAs) respectively corresponding to the first plurality of DNAs of step (a) via in vitro transcription;
(c) mixing the first plurality of D-RNAs of step (b) with a first plurality of streptavidin-coated magnetic beads, which are pre-treated with transfer RNAs (tRNAs);
(d) collecting and mixing the supernatant of the product of step (c) with a biotinylated L-form nucleic acid having the nucleotide sequence of “GGGGCGGGUGGGGAGGGG” (SEQ ID NO: 4);
(e) mixing the product of step (d) with a second plurality of streptavidin-coated magnetic beads, which are pre-treated with the tRNAs;
(f) eluting the precipitate of step (e) with an elution buffer so as to produce a second plurality of D-RNAs specific to the L-form nucleic acid;
(g) producing a plurality of complementary DNAs (cDNAs) respectively corresponding to the second plurality of D-RNAs of step (f) via reverse transcription;
(h) producing a second plurality of DNAs respectively corresponding to the plurality of cDNAs of step (g) via polymerase chain reaction (PCR);
(i) repeating steps (b) to (h) at least 4 times, in which the in vitro transcription was performed by using the product of step (h), so as to identify a D-RNA aptamer specific to the L-form nucleic acid; and
(j) producing the L-RNA aptamer corresponding to the D-RNA aptamer of step (i).

Each randomized sequence of the DNA library is composed of adenine (A), thymine (T), cytosine (C), and guanine (G) nucleotides. Preferably, the ratio of the sum of the A and T nucleotides to the sum of the C and G nucleotides in each randomized sequence is about 7:3.

Optionally, the present method further comprises (c-1) subjecting the product of step (c) to centrifugation or magnetic field prior to step (d).

Still optionally, the present method further comprises (e-1) subjecting the product of step (e) to centrifugation or magnetic field prior to step (f).

Also disclosed herein is a recombinant polynucleotide derived from the present L-RNA aptamer. According to some embodiments of the present disclosure, the recombinant polynucleotide is characterized by having the loop region of the L-RNA aptamer (i.e., “UGGGAAAGGGU”; SEQ ID NO: 2). In these embodiments, the recombinant polynucleotide comprises the nucleotide sequence of “GGCGCACUGGCGCUGCGCCUGGGAAAGGGUGGCGCCAAUCGUAGCGUGUCGGCGCC” (SEQ ID NO: 3).

Another aspect of the present disclosure pertains to a method of treating Alzheimer's disease in a subject. The method comprises administering to the subject an effective amount of the L-RNA aptamer of the present disclosure so as to alleviate or ameliorate the symptoms associated with Alzheimer's disease.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings.

FIG. 1A is a schematic diagram depicting the predicted secondary structures of aptamers Apt. 1 according to Example 1 of the present disclosure.

FIG. 1B is a photograph of a gel depicting the binding between D-aptamer 1 (D-Apt.1) and FAM APP L-rG4 target in electrophoretic mobility shift assay (EMSA) according to Example 1 of the present disclosure.

FIG. 2A is a schematic diagram depicting the predicted secondary structures of aptamers Apt. 1-1 according to Example 2 of the present disclosure.

FIGS. 2B and 2C respectively depict the EMSA results according to Example 2 of the present disclosure. FIG. 2B: a photograph of a gel depicting the binding between D-Apt. 1-1 and FAM APP L-rG4 target in EMSA. FIG. 2C: the binding curve of D-Apt. 1-1 to FAM APP L-rG4 obtained from EMSA result of FIG. 2B.

FIG. 3 depicts the binding specificity of L-Apt. 1-1 towards specified construct according to Example 3 of the present disclosure.

FIGS. 4A and 4B respectively depict the effect L-Apt. 1-1 on the endogenous APP level in cells according to Example 3 of the present disclosure. FIG. 4A: a photograph of western blot depicting the endogenous APP level treated with L-Apt. 1-1 or D-Apt.1-1 (0, 25, 50 or 100 nM); GAPDH was used as loading control. FIG. 4B: Quantification of endogenous APP protein level relative to GAPDH. Values were obtained from three replicates and error bars display standard error of mean. *, p<0.05. NS, not significant.

FIG. 5 is a histogram depicting the fold effects of bi-functional FLAPs with or without APP L-rG4 having different transducer lengths at P3 stem according to Example 4 of the present disclosure.

FIG. 6 is a histogram depicting the quantification of the overlap ratio of L-Apt. 1-1_Pepper foci and APP rG4 foci for wild-type (wt) and mutant (mut) groups according to Example 4 of the present disclosure. About 750 foci for wt group and 1000 foci for mut group were counted. Values were obtained from three replicates and error bars display standard error of mean.

In accordance with common practice, the various described features/elements are not drawn to scale but instead are drawn to best illustrate specific features/elements relevant to the present invention.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

As used herein, the term “L-form RNA” (L-RNA) refers to an artificial RNA built from L-ribose. Compared to naturally occurring oligonucleotides (e.g., D-form RNA, D-RNA), which are homochiral and are built from D-ribose, L-RNA is an enantiomeric counterpart of the natural oligonucleotide and is artificially synthesized via chemical reactions by using L-ribose as the major stating material.

As used herein, the term “aptamer” refers to an oligonucleotide (e.g., a DNA or RNA oligonucleotide) having specific binding regions which are capable of forming complexes with an intended target molecule in an environment wherein other substances in the same environment are not complexed to the oligonucleotide.

The term “G-quadruplex structure” (G4 structure) as used herein refers to a four-stranded helical nucleic acid structure comprising multiple stacked G-tetrads, each of which consists of four guanine bases that associate in a cyclical manner through Hoogsteen hydrogen bonds and are further stabilized, through coordination to a cation in the center. The body of stacked G-tetrads, comprising a total of 2-8 layers, is collectively referred to as the G-tetrad core. Each of the four guanine columns constituting the G-tetrad core can arise from a single (continuous column), two, or four (discontinuous column) separate guanine stretch/stretches. The term “parallel G-quadruplex”, as used herein, relates to a G-quadruplex structure wherein all four strands point in the same direction.

As used herein, the terms “polynucleotide” and “nucleic acid” are interchangeable, and refers to a polymer of nucleotides (ribonucleotides, deoxynucleotides, and/or the derivatives or analogs thereof) having at least about 10 bases or base pairs in length. The term “polynucleotide” includes single and double stranded forms of DNA. As used herein, the term “recombinant polynucleotide” refers to a polynucleotide produced by artificially recombinant DNA techniques, wherein the nucleotide sequence of the polynucleotide is not identical to that of any naturally occurring sequence.

The term “transcription” is known in the art, and refers to a reaction during which a nucleic acid molecule (e.g., a DNA molecule) serving as a template and having a particular nucleotide sequence is read by an RNA polymerase so that the RNA polymerase produces a single-stranded RNA molecule. During transcription, the genetic information in a nucleic acid template is transcribed, and subsequently the transcribed RNA may be further translated into protein. As used herein, the term “in vitro transcription” refers to a reaction wherein RNA, in particular mRNA, is in vitro synthesized from DNA template in a cell-free system, e.g., appropriate cell extracts. According to some embodiments of the present disclosure, in vitro transcription is carried out by incubating reactants, including DNA template containing a promoter, RNA polymerase, nucleotide triphosphates (NTPs), reaction buffer (e.g., Tris or Tris-HCl), salt (e.g., MgCl₂), reducing agent (optionally; e.g., dithiothreitol, DTT) and RNase inhibitor (optionally), at an appropriate temperature (e.g., 37° C.) for a period of time.

As used herein, the term “reverse transcription” refers to a reaction in which an RNA template is reverse transcribed using a reverse transcriptase into a complementary DNA (cDNA) chain. A reverse transcription reaction usually includes, RNA template, reverse transcriptase, reaction buffer (e.g., Tris or Tris-HCl), salt, primers (e.g., oligo(dT) primers or random primers), deoxynucleoside triphosphates (dNTPs), reducing agent (optionally; e.g., dithiothreitol, DTT) and RNase inhibitor (optionally). Depending upon the context, the mixture can be either a complete or incomplete reverse transcription reaction mixture.

The terms “administered” and “administering” are used interchangeably herein to refer a mode of delivery, including, without limitation, intrathecal, intra-cerebellar, intratumoral, intravenous, intraarterial, intraperitoneal, or subcutaneous injection of an agent (e.g., the L-RNA aptamer) of the present invention.

As used herein, the term “treat,” “treating” and “treatment” are interchangeable, and encompasses partially or completely preventing, ameliorating, mitigating and/or managing a symptom, a secondary disorder or a condition associated with Alzheimer's disease. The term “treating” as used herein refers to application or administration of the L-RNA aptamer of the present disclosure to a subject, who has a symptom, a secondary disorder or a condition associated with Alzheimer's disease, with the purpose to partially or completely alleviate, ameliorate, relieve, delay onset of, inhibit progression of, reduce severity of, and/or reduce incidence of one or more symptoms, secondary disorders or features associated with Alzheimer's disease. Symptoms, secondary disorders, and/or conditions associated with Alzheimer's disease include, but are not limited to, memory loss, poor judgment, loss of spontaneity and sense of initiative, repeating questions, forgetting recently learn information, mood and personality changes, increased anxiety and/or aggression, and/or difficulty completing tasks (e.g., bathing). Treatment may be administered to a subject who exhibits only early signs of such symptoms, disorder, and/or condition for the purpose of decreasing the risk of developing the symptoms, secondary disorders, and/or conditions associated with Alzheimer's disease. Treatment is generally “effective” if one or more symptoms or clinical markers are reduced as that term is defined herein. Alternatively, a treatment is “effective” if the progression of a symptom, disorder or condition is reduced or halted.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives.

The term “subject” refers to a mammal including the human species that is treatable with the L-RNA aptamer and/or method of the present invention. The term “subject” is intended to refer to both the male and female gender unless one gender is specifically indicated.

### II. Description of the Invention

### (i) L-RNA Aptamer and Uses Thereof in Treating Diseases

The first aspect of the present disclosure is directed to an L-RNA aptamer exhibiting binding affinity and specificity towards 3′-UTR of APP mRNA. According to some embodiments of the present disclosure, the L-RNA aptamer designated as “L-Apt.1-1” is characterized by having a loop region that recognizes and binds to target sequence (i.e., the rG4 structure at 3′-UTR of APP mRNA; hereinafter as “APP rG4”), and upstream and downstream regions respectively disposed at the 5′ end and 3′ end of the loop region that form the stem domain holding the aptamer structure into a hairpin structure. In these embodiments, the L-Apt.1-1 comprises the nucleotide sequence of “GUGUAUAGAUGGGAAAGGGUUCUAUACGCA” (SEQ ID NO: 1), in which the loop region is in bold font, and the upstream and downstream regions are in italic font.

According to certain embodiments of the present disclosure, the L-Apt. 1-1 is produced by a method comprising the steps of,
(a) providing a low GC-content DNA library;
(b) producing a plurality of D-RNAs (hereinafter as “low GC-content D-RNAs”) respectively corresponding to the low GC-content DNAs via in vitro transcription;
(c) mixing the low GC-content D-RNAs of step (b) with streptavidin-coated magnetic beads, which are pre-treated with tRNAs;
(d) collecting and mixing the supernatant of the product of step (c) with a biotinylated L-form target nucleic acid;
(e) mixing the product of step (d) with streptavidin-coated magnetic beads, which are pre-treated with the tRNAs;
(f) eluting the precipitate of step (e) with an elution buffer so as to produce a plurality of D-RNAs specific to the L-form target nucleic acid (hereinafter as “target-specific D-RNAs”);
(g) producing a plurality of cDNAs respectively corresponding to the target-specific D-RNAs of step (f) via reverse transcription;
(h) producing a plurality of DNAs respectively corresponding to the plurality of cDNAs of step (g) via PCR;
(i) repeating steps (b) to (h) at least 4 times, in which the in vitro transcription was performed by using the product of step (h), so as to identify a D-RNA aptamer specific to the L-form target nucleic acid; and
(j) producing the L-RNA aptamer corresponding to the D-RNA aptamer of step (i).

In the step (a), a low GC-content DNA library comprising a plurality of DNAs (hereinafter as “low GC-content DNAs”) is provided. According to some embodiments of the present disclosure, each of the low GC-content DNAs has a randomized nucleotide sequence composed of 30 nucleotides (i.e., thirty A, T, C, and/G nucleotides) in length (N30). Preferably, the ratio of the sum of A and T nucleotides to the sum of C and G nucleotides in each low GC-content DNA is about 7:3, i.e., the numbers of A and T nucleotides: the numbers of C and G nucleotides=7:3. In certain embodiments, the percentages of A, T, C, and G nucleotides present in the low GC-content DNA are respectively 35%, 35%, 15%, and 15%.

According to some optional embodiments of the present disclosure, the L-RNA aptamer is produced from a low GC-content single-stranded DNA (ssDNA) library. In this case, the method further comprises the step of producing the low GC-content DNA library from the low GC-content ssDNA library via reverse transcription prior to step (a). According to one exemplary embodiment, the low GC-content DNA library is produced by primer extension on the ssDNA of the ssDNA library with reverse primer, in the presence of reverse transcriptase, dNTPs (including dATP, dTTP, dCTP and dGTP) and reaction buffer (e.g., Tris-HCl). Preferably, the reaction buffer contains one or more salts (e.g., MgCl₂ and/or LiCl) and/or reducing agent (e.g., DTT) to improve the transcription efficiency.

In the step (b), the low GC-content DNA library is subjected to in vitro transcription so as to produce low GC-content D-RNAs. According to some embodiments, the in vitro transcription is carried out by mixing low GC-content DNAs (serving as DNA templates) with RNA polymerase (e.g., T7, SP6 or T3 RNA polymerase), NTPs (including ATP, UTP, CTP and GTP) and a reaction buffer (e.g., T7, SP6 or T3 reaction buffer), followed by incubating at an appropriate temperature (e.g., 37° C.) for a period of time (e.g., 2-3 hours). Optionally, the reaction buffer may contain one or more salts (e.g., MgCl₂), reducing agent (e.g., dithiothreitol, DTT), RNase inhibitor or a combination thereof, so as to improve the transcription efficiency. A skilled artisan may adjust the reactants, their concentrations and reaction parameters (e.g., temperature and reaction time) in accordance with intended purposes. The thus-produced low GC-content D-RNAs respectively have the nucleotide sequences corresponding to the low GC-content DNAs.

In the step (c), the low GC-content D-RNAs of step (b) are subjected to a negative selection via mixing with streptavidin-coated magnetic beads (e.g., streptavidin-coated iron beads). According to preferred embodiments of the present disclosure, the streptavidin-coated magnetic beads are pre-treated with tRNA to inhibit non-specific binding sites.

Optionally, the mixture of the magnetic beads and low GC-content D-RNAs as described in step (c) is subjected to centrifugation or magnetic field so as to separate beads-bound D-RNAs from non-bound D-RNAs (step (c-1)). According to certain embodiments, the mixture of step (c) is centrifuged at low speed (e.g., 100, 200, 300, 400 or 500 rpm) for a period of time (e.g., 1-2 hours) to precipitate the magnetic beads and beads-bound D-RNAs (i.e., D-RNAs which bind to magnetic beads without specificity). According to alternative embodiments, the mixture of step (c) is exposed to a magnetic field to achieve the same purpose.

Then, in the steps (d) and (e), the supernatant of the product of step (c), which contains non-bound D-RNAs, is subjected to a positive selection so as to identify the D-RNAs having binding affinity to the target nucleic acid. In practice, the supernatant of step (c) is collected and then mixed with a biotinylated target nucleic acid, i.e., a target DNA or RNA sequence having a biotin molecule conjugated at its 5′- or 3′-end (step (d)). In certain embodiments, the biotinylated target nucleic acid is a biotinylated APP L-rG4 having the nucleotide sequence of “GGGGCGGGUGGGGAGGGG” (SEQ ID NO: 4). After incubating for a period of time (e.g., 30-60 minutes), the mixture containing non-bound D-RNAs and biotinylated target nucleic acid is further mixed with streptavidin-coated magnetic beads (e.g., streptavidin-coated iron beads), which are pre-treated with tRNA to inhibit non-specific binding sites (step (e)). In this case, the D-RNAs having binding affinity to the target nucleic acid can be isolated from the mixture via the interaction between the complementary sequences of biotinylated target nucleic acid (e.g., biotinylated APP L-rG4) and the D-RNA, and the streptavidin-biotin interaction between the biotinylated target nucleic acid and streptavidin-coated magnetic beads.

Optionally, the mixture of step (e) is subjected to centrifugation or magnetic field so as to separate beads-bound D-RNAs from non-bound D-RNAs (step (e-1)). According to certain embodiments, the mixture of step (e) is centrifuged at low speed (e.g., 100, 200, 300, 400 or 500 rpm) for a period of time (e.g., 1-2 hours) thereby precipitating the magnetic beads and beads-bound D-RNAs (i.e., the D-RNAs bound to magnetic beads via interacting with the biotinylated target nucleic acid). According to alternative embodiments, the mixture of step (e) is exposed to a magnetic field to achieve the same purpose.

In the step (f), the D-RNAs bound to the magnetic beads are eluted with the aid of an elution buffer, which disrupts the binding of the D-RNAs, biotinylated target nucleic acid (e.g., biotinylated APP L-rG4) and magnetic beads. According to some embodiments of the present disclosure, the elution buffer is an alkaline solution containing a chelating agent, for example, ethylenediaminetetraacetic acid (EDTA).

Next, in the step (g), reverse transcribing the target-specific D-RNAs produced in step (f) into cDNAs. The methods and conditions for reverse transcribing an RNA template into its corresponding cDNA are known by the person having ordinary skill in the art; for the sake of brevity, the detailed descriptions are omitted herein. According to some working examples of the present disclosure, the reverse transcription is carried out by mixing the target-specific D-RNAs with reverse transcriptase, reverse primers, dNTPs (including dATP, dTTP, dCTP and dGTP) and reaction buffer (e.g., Tris-HCl), and then incubated at an appropriate temperature (e.g., 50° C.) for a period of time (e.g., 30 minutes). Depending on desired purposes, the reaction buffer may contain one or more salts (e.g., MgCl₂ and/or LiCl), reducing agent (e.g., DTT), RNase inhibitor or a combination thereof, thereby improving the transcription efficiency. A skilled artisan may adjust the reactants, their concentrations and reaction parameters (e.g., temperature and reaction time) in accordance with intended purposes. The thus-produced cDNAs respectively have the nucleotide sequences corresponding to the target-specific D-RNAs.

Optionally, the present method further comprises adding an alkaline solution (e.g., NaOH) to the cDNAs of step (g), followed by heating the mixture for a period of time (e.g., heating the mixture at 95° C. for 10 minutes) to degrade RNA and protein (step (g-1)). Then, the cDNAs may optionally be neutralized by an acidic solution (e.g., Tris-HCl, pH7.5) and/or purified by a column.

In the step (h), double-stranded DNAs (dsDNAs) corresponding to the cDNAs of step (g) are produced by PCR. The steps and conditions for producing dsDNAs from cDNAs via PCR are known by the person having ordinary skill in the art; for the sake of brevity, the detailed descriptions are omitted herein. According to certain working examples of the present disclosure, the PCR is carried out by mixing the cDNAs of step (g) (serving as templates) with DNA polymerase, forward and reverse primers, dNTPs (including dATP, dTTP, dCTP and dGTP) and reaction buffer (e.g., Tris-HCl). Depending on intended purposes, the reaction buffer may contain one or more salts (e.g., MgCl₂).

As described in the step (i), the dsDNAs produced by step (h) may serve as the transcription templates of the next round of selection, i.e., the templates for in vitro transcription of step (b). According to some embodiments, steps (b) to (h) are repeated at least 4 times (e.g., 4, 5, 6, 7, 8, 9, 10 or more times) so as to improve the binding specificity of the selected product (i.e., the target-specific D-RNA and its corresponding cDNA and dsDNA) to the target nucleic acid. According to one exemplary embodiment of the present disclosure, for the purpose of identifying the aptamer L-Apt. 1-1, steps (b) to (h) are repeated 5 times. In these embodiments, the target-specific D-RNA produced by step (f) of the last round of selection exhibits binding affinity and/or specificity towards the target nucleic acid, and is identified as the D-RNA aptamer.

Optionally, the method further comprises subjecting the DNA produced by step (h) of the last round of selection to sequencing technology, for example, next generation sequencing (NGS) or Sanger sequencing, so as to identify the nucleotide sequences of the DNA and its corresponding cDNA and D-RNA aptamer.

Finally, in the step (j), the L-RNA aptamer (i.e., aptamer L-Apt.1-1) is chemically synthesized (e.g., solid-phase synthesis) via using the nucleotide sequence of the identified D-RNA aptamer of step (i) as a template.

As could be appreciated, the aptamer L-Apt. 1-1 of the present invention may alternatively be produced by in vitro transcription. A skilled artisan may produce the aptamer L-Apt. 1-1 via a suitable method in accordance with the nucleotide sequence described in the present disclosure.

According to certain working examples, the aptamer L-Apt. 1-1 does not contain rG4 motif, and exhibits binding affinity and specificity towards APP rG4. According to some working examples, the aptamer L-Apt. 1-1 could inhibit the translation of APP.

The second aspect of the present disclosure is thus directed to a method of treating Alzheimer's disease by using the present aptamer L-Apt. 1-1. The method comprises administering to a subject in need thereof an effective amount of the aptamer L-Apt. 1-1 to alleviate or ameliorate the symptoms associated with Alzheimer's disease.

Depending on intended purpose, the aptamer L-Apt. 1-1 may be administered to the subject via any suitable route, for example, oral, enteral, nasal, transmucosal, intravenous, intraarterial, intramuscular, subcutaneous, intrathecal, intraperitoneal, or intra-cerebellar injection.

### (ii) Recombinant Polynucleotide and Uses Thereof in Detecting APP rG4

Based on the binding affinity and specificity towards APP rG4, the present aptamer L-Apt. 1-1 may be conjugated with a reporter molecule (for example, a fluorescent RNA, FR) for detecting APP rG4 structure in vitro and in vivo. Thus, the third aspect of the present disclosure pertains to a recombinant polynucleotide that incorporates the loop sequence (i.e., SEQ ID NO: 2) of the present aptamer L-Apt. 1-1 into the nucleotide sequence of a fluorescent RNA. As aforementioned, the loop region of the aptamer L-Apt. 1-1 accounts for target sequence (i.e., APP rG4) recognition, while the upstream and downstream regions flanking the loop region only hold the aptamer structure into a hairpin structure. In this case, the L-RNA-based recombinant polynucleotide may serve as a fluorogenic bi-functional aptamer (FLAP) that recognizes and binds to the target sequence via the loop region of the aptamer L-Apt. 1-1, while binds and activates fluorescent dyes via the fluorescent RNA.

Depending on desired purpose, the fluorescent RNA may be any fluorogenic aptamer known in the art, for example, Pepper aptamer, Spinach aptamer, Mango aptamer, Broccoli aptamer, Corn aptamer, Chili aptamer, and etc. The structure and function of the fluorogenic aptamer are known by a skilled artisan in the art; for example, see, Lynda Truong et al., Protein Society, 2019, 28:1374-1386; Robert J. Trachman III et al., Trends in Pharmacological Sciences, 2017, 38:928-939; or Xianjun Chen et al., Nature Biotechnology, 2019, 37:1287-1293. Hence, the detailed descriptions thereof are omitted herein for the sake of brevity.

According to some exemplary embodiments of the present disclosure, the fluorogenic aptamer is Pepper aptamer. In these embodiments, the loop sequence of the aptamer L-Apt. 1-1 is incorporated into P1 or P3 stem sequence of the Pepper aptamer. According to certain embodiment, the loop sequence of the aptamer L-Apt. 1-1 is incorporated into P3 stem sequence of the Pepper aptamer via a linker sequence having 1, 2, 3, 4, or 5 base pairs (bp). Preferably, the linker sequence has 5 base pairs; in this case, the thus-produced recombinant polynucleotide designated as “L-Apt. 1-1_Pepper” comprises the nucleotide sequence of “GGCGCACUGGCGCUGCGCCUGGGAAAGGGUGGCGCCAAUCGUAGCGUGUCGGCGCC” (SEQ ID NO: 3), in which the loop sequence of aptamer L-Apt. 1-1 is in bold font.

As described above, the L-Apt. 1-1_Pepper of the present invention may be produced by in vitro transcription or chemical synthesis (e.g., solid-phase synthesis). A skilled artisan may produce the L-Apt. 1-1_Pepper via a suitable method in accordance with the nucleotide sequence described in the present disclosure.

According to certain embodiment, the L-Apt. 1-1_Pepper is useful in detecting APP rG4 in the presence of HBC530 ligand (a mimic of green fluorescent protein (GFP) fluorophore).

Also disclosed herein is a method of detecting the APP rG4 in a biological sample. The method comprises,
(a) adding the present recombinant polynucleotide (e.g., L-Apt.1-1_Pepper) to the biological sample in the presence of a suitable fluorescent dye (e.g., HBC530);
(b) irradiating a light having a suitable excitation wavelength to excite the product of step (a); and
(c) measuring the fluorescence emitted from the product of step (b) thereby detecting the APP rG4 in the biological sample.

As could be appreciated, the excitation wavelength of step (b) varies with the types of fluorescent dye and fluorogenic aptamer. For example, in the case when the fluorogenic aptamer is Pepper aptamer and the fluorescent dye is HBC530, then the excitation wavelength used to excite the Pepper aptamer/HBC530 ranges from about 450 nm to about 500 nm.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

In Vitro Selection (rG4-Systematic Evolution of Ligands by Exponential Enrichment, rG4-SELEX-Seq)

G4-SELEX-seq started with the extension of a low GC ssDNA N30 random library (each of the ssDNA had a randomized nucleotide sequence composed of 30 nucleotides in length with A:T:G:C ratio of 35%:35%:15%:15%) to dsDNA library. The ssDNA library (2 μM) was mixed with reverse primer (3 μM), 4 μL of 5× Li reverse transcriptase (RT) buffer and dNTPs mix (100 mM Tris-HCl pH 7.5, 20 mM MgCl₂, 5 mM DTT, 750 mM LiCl and 5 mM dNTPs) in nuclease-free water (19 μL), and heated at 95° C. for 5 minutes. After cooling down, 1 μL of 200 U/μL reverse transcriptase was added to the mixture and incubated at 50° C. for 50 minutes. The dsDNA library generated (100-150 ng) was then in vitro transcribed into N30 random RNA library with RNA synthesis kit with 40 μL volume, followed by the removal of DNA template by adding 2 μL DNase. Next, the transcribed RNAs were purified by a 10% denaturing polyacrylamide gel electrophoresis (PAGE) and collected by using RNA clean and concentrator column.

To prepare for selection, 75 μL of MyOne™ streptavidin C1 dynabeads at 10 mg/mL was washed with 600 of μL solution A (100 mM NaOH and 50 mM NaCl) and 600 μL of solution B (100 mM NaCl), and resuspended in 75 μL of 2× selection buffer (150 mM KCl, 25 mM Tris-HCl pH 7.5 and 10 mM MgCl₂). Then, 0.75 μL of 10 mg/mL yeast tRNA was added to the mixture with shaking at 700 rpm for 30-45 minutes at 25° C. using a thermo-shaker. After that, the RNA library pool with a final concentration of 4.5 μM was mixed with 2×RNA buffer (300 mM KCl and 50 mM Tris-HCl pH 7.5) to 50 μL, then refolded by heating at 95° C. and cooling on ice. 25 μL tRNA pre-blocked beads were added and incubated at 700 rpm for 2 hours. Afterward, the beads were discarded to remove non-specific binding RNAs.

To start the positive selection, 5′ biotin labelled APP L-rG4 was refolded by heating at 95° C. and cooling on ice, and mixed with the RNA pool with a final concentration of 0.65 μM. The resultant mixture was shaken at 300 rpm for 30 minutes for target binding. After that, the biotin-labelled APP L-rG4 and RNA complexes were pulled down using 50 μL of beads, and washed with 600 μL of 1× selection buffer for 5 times.

Lastly, target binding RNAs were eluted with 100 μL of elution buffer (25 mM NaOH and 1 mM EDTA), and neutralized by 50 μL of 1M Tris-HCl immediately. The collected RNAs were then recovered to 30 μL using RNA clean and concentrator column. To generate ssDNA template from the selected RNAs by reverse transcription, the 30 μL purified RNA was mixed with reverse primer (0.33 μM), and heated at 95° C. for 5 minutes, then cooled down at 35° C. for 10 minutes. Then, 18 μL 5× Li RT buffer and 5 mM dNTPs mix, and 2 μL of 200 U/μL RT were added, and diluted with nuclease-free water to 60 μL, and incubated at 50° C. for 15 minutes. The reaction was quenched by adding 0.6 μL of 10 M NaOH, and heated at 95° C. for 10 minutes to degrade the RNAs. After that, the resultant ssDNA was cooled to 25° C., and neutralized by 15 μL of 1 M Tris-HCl pH 7.5. The reverse transcribed ssDNA was recovered to 30 μL using RNA clean and concentrator column and PCR amplified for next round selection. A small-scale PCR cycle test was carried out to decide the optimum number of cycles for large-scale DNA amplification. Forward primer and reverse primer (0.5 μM each), 8 μL of ssDNA and 10 μL of 2×KAPA HIFI™ HotStart ReadyMix were mixed to a total of 20 μL, and divided into 5 aliquots of 4 μL each. The aliquots were PCR amplified for 8, 10, 12, 14 and 16 cycles, respectively (3 minutes at 98° C., [20 seconds at 98° C., 20 seconds at 60° C., 20 seconds at 72° C.]ₙ, 3 minutes at 72° C., 4° C.). The optimum number of cycles was determined by the fewest PCR cycles that provided a clear band on an agarose gel stained by 6×DNA loading dye and DNA gel stain. Lastly, the large-scale DNA amplification was carried out with 40 μL reaction volume with the same condition, and the dsDNA product was purified and collected using DNA recovery kit. After five rounds of selection, the dsDNAs were amplified for 4 cycles with NGS primers and dehydrated to vacuum concentration with a total of >200 ng. The dsDNAs were then submitted for NGS sequencing, and the results for 2-5 rounds were reported and analyzed by software.

### Electrophoretic Mobility Shift Assay (EMSA)

Four-fold serial dilution was performed for D- or L-Apt. 1-1 with an initial concentration of 8 μM to generate 12 sets of sample solution in selection buffer containing 25 mM Tris-HCl (pH 7.5), 150 mM KCl, 1 mM MgCl₂, with the addition of 8% glycerol or sucrose solution and 20 nM 5′ FAM labelled APP L- or D-rG4. The aptamer solutions were refolded by heating at 95° C. and cooling on ice, then mixed with APP rG4 and incubated at 37° C. for 30 minutes. The solutions were then loaded to 10% native non-denaturing gel in 50 mM potassium acetate, 25 mM Tris-HCl pH 7.5 and 1 mM MgCl₂ buffer, and conducted at 70 mA for 50 minutes at 4° C. The binding was visualized by scanning the gel with an image scanner, followed by gel image quantification and analysis using software. For magnesium dependence, all conditions were the same except for MgCl₂ concentration (0 and 2 mM). For binding test, sample solutions containing 0, 20, 200 and 2000 nM aptamer candidates and 10 nM 5′ FAM labelled D-target were prepared, and other procedures were the same. For mutagenesis analysis, sample solutions containing 100 nM D-Apt.1-1 wildtype and mutant, and 10 nM 5′ FAM labelled APP L-rG4 were prepared, and other procedures were the same. For binding specificity, sample solutions containing 100 nM L-Apt. 1-1 and 5/10 nM 5′ FAM labelled D-target were prepared, and other procedures were the same.

### Circular Dichroism Spectroscopy

Sample solutions containing 5 μM D-Apt. 1-1, 10 mM LiCac pH 7.0 and 150 mM KCl or LiCl were prepared, and were refolded by heating at 95° C. and cooling on ice. Samples were vortexed and spun down before transferring to the quartz cuvette and scanned for 2 times at 25° C. within 220 and 310 nm by using circular dichroism spectrophotometer. The data was obtained in every 1 nm with 0.5 s/nm responding time, and were normalized and smoothed over 5 nm to generate the spectrum using software. The figure was then generated using software.

### G4-Ligand Enhanced Fluorescence Assay

Sample solutions containing 1 μM D-Apt.1-1, 10 mM LiCac pH 7.0, 150 mM KCl/LiCl were prepared, and were refolded by heating at 95° C. and cooling on ice. Thioflavin T (ThT) or N-methyl mesoporphyrin IX (NMM) ligands were added to the solutions and transferred to the quartz cuvette. The excitation wavelength were 425 nm and 394 nm for ThT and NMM, respectively, and the emission fluorescence were measured in the range of 440-700 nm for ThT and 550-750 nm for NMM at 25° C. using fluorometer. The experiment was conducted with 5 nm entrance slit, 2 nm exit slit and 2 nm increment. The data was then processed and plotted using software.

### Biostability Assay

Sample solutions containing 100 nM D- or L-Apt.1-1 were refolded in selection buffer by heating at 95° C. and cooling on ice, then incubated at 25° C. for 2 hours. In the period, 100 ng/μL RNase A or 5% fetal bovine serum (FBS) were added to each sample solution at 0 minutes and every 30 minutes, respectively. The RNAs were then visualized by a 6% denaturing PAGE with SYBR™ gold nucleic acid gel stain, scanned by an image scanner.

### Western Blotting

1×10⁵/well Hela cells were seeded onto a 24-well plate. The next day, cells were transfected with different concentrations of L-Apt. 1-1 or D-Apt. 1-1 by LIPOFECTAMINE™ 2000. After about 48 hours post-transfection, cells were harvested and lysed in TRITON® X-100 lysis buffer (50 mM Tris-HCl pH 7.5, 1% TRITON® X-100, 250 mM NaCl, 5 mM EDTA) supplemented with protease inhibitor cocktail. Cell lysates were centrifuged at 16,000 g for 10 miutesn at 4° C. and the supernatants were boiled with sample buffer for 10 minutes. Then samples were resolved by 8% sodium dodecyl sulfate polyacrylamide gel electrophoresis (SDS-PAGE) and transferred to polyvinylidene difluoride (PVDF) membrane. Blocking was done by incubating the membrane with 5% milk in TBST (Tris-buffered saline with 0.1% TWEEN® 20 detergent) for 1 hour with rotation. Then, the membrane and the primary antibody (APP, 1:1,000) (GAPDH, 1:1,000) were mixed and shaken for 1 hour. The membrane was washed for 4 times using TBST with rotation, 8 minutes each time. After that, horseradish peroxidase (HRP)-conjugated secondary antibody was added to the membrane and shaken for 1 hour. After 4 times washing with TBST, 5 minutes each time, the membrane was treated with substrates and scanned by an imaging system. Protein band intensity was then analyzed by software.

Quantitative Reverse Transcription-Polymerase Chain Reaction (qRT-PCR)

Total RNAs were extracted using extraction kit. 80 ng of total RNAs were reverse transcribed to cDNA using RT. For qRT-PCR detection, TB Green® Premix Ex Taq™ was used together with primers of APP and GAPDH separately. GAPDH was an internal control. Relative mRNA level of APP was obtained from normalization by GAPDH.

### Characterization of Apt. 1-1_Pepper

Samples containing 0.5 μM D- or L-Apt. 1-1_Pepper were refolded by heating at 95° C. and cooling on ice in selection buffer. Various concentrations of L- or D-rG4 were then added and incubated at 37° C. for 30 minutes, respectively. Then, 1 μM HBC530 (an RNA mimic of GFP fluorophore) was added and incubated at 37° C. for 15 minutes. Sample solutions were loaded into 384 well black plate and scanned with microplate reader with excitation at 455 nm and emission at 495-650 nm. The data was then processed and plotted using software.

### Cell Imaging of APP rG4 Using L-Apt. 1-1_Pepper

2.5×10⁴ Hela or HEK293T cells were seeded onto a 35 mm confocal dish for 24 hours. Next day, TAMRA-wt/mut APP rG4 motif oligo was refolded in selection buffer containing 25 mM Tris-HCl (pH 7.5), 150 mM KCl, 1 mM MgCl₂, by heating at 95° C. and cooling at 25° C. for 5 minutes and at 4° C. for at least 5 minutes, respectively. Then, Hela cells were transfected with 200 nM refolded TAMRA-wt/mut APP rG4 motif oligo, while HEK293T cells with 100 nM oligos, respectively using LIPOFECTAMINE™ 2000. After about 18 hours, cells were washed with phosphate buffered saline (PBS) for one time, fixed with 4% paraformaldehyde at 25° C. for 15 minutes and washed with 2× saline sodium citrate (SSC) or PBS for 5 times. The cells were then permeabilized by 0.5% TRITON® X-100 at 37° C. for 30 minutes and washed with 2×SSC for 4 times. L-Apt.1-1_Pepper was refolded in buffer containing 25 mM Tris-HCl (pH 7.5), 150 mM KCl, 5 mM MgCl₂, by heating at 95° C. for 5 minutes and cooling at 4° C. for 5 minutes. Next, 500 nM refolded L-Apt.1-1_Pepper and 2 μM HBC530 were added to the dish in stain buffer containing 30% formamide, 10% dextran sulfate, 2.4×SSC, 5 mM MgCl₂, to stain cells overnight at 37° C. Next day, cells were washed with 2×SSC for 5 times and stained with 5 μg/ml Hoechst 33342 overnight at room temperature. At last, cells were scanned by confocal microscopy. The percentage of colocalized green and red was calculated and processed using software.

### Example 1 Identification of a Novel Non-rG4-Containing D-RNA Aptamer that Binds APP L-rG4

In G4-SELEX-seq, the G4-SELEX part involved stereospecific enzymes that could only recognized D-form nucleic acids, thus the chirality of the library pool and APP rG4 target were reversed to begin the first selection cycle with a D-library pool and biotin-APP L-rG4 target. In order to increase the probability of successfully obtaining non-G4-containing aptamer candidates, a low GC-content library pool was introduced to minimize the enrichment of G-rich sequences that may promote G4 formation in the initial G4-SELEX round. The library pool was designed with randomized low GC 30 nt region, flanked by primer binding sequences at 5′ and 3′ ends with T7 promoter sequence included. The DNA library was transcribed to RNA library for selection. Through the SELEX cycles, non-target binding sequences were discarded, while the biotinylated target-aptamer complexes were pulled down by tRNA-blocked streptavidin-coated magnetic beads. The target-binding aptamer candidates were then eluted and reverse transcribed for PCR amplification. To select for stronger binding aptamer candidates, the SELEX cycles were then repeated with increasing stringency of selection conditions in subsequent rounds. After five rounds of selection, the aptamer candidates were sequenced by next generation sequencing (NGS).

From the NGS results, enrichment of specific sequences was observed (data not shown). With close inspection of the sequence data, it was found that there was an enriched conserved region 5′-TGGGAAAGGGT-3′ (SEQ ID NO: 5) among the aptamer candidates in the NGS result (data not shown). To shortlist candidates for downstream experiments, the Mfold program was used to predict the secondary structures of aptamer candidates with or without linker regions. It is worth noting that Mfold cannot predict G4 structure formation, so G4 prediction analysis was also performed using G4RNAscreener. The G4 prediction scores indicated that most aptamer candidates are unfavorable in forming G4 structures (data not shown). Considering the sequence enrichment and percentage of reads, consistency of the secondary structures with or without linkers, and the low G4 prediction scores, six Apts (including Apt. 1, 2, 4, 6, 9, and 13) were selected for an initial binding test by electrophoretic mobility shift assay (EMSA). The results indicated that Apt. 1, 2, 4, 6, 9 exhibited strong binding with 5′ FAM APP L-rG4 target compared with Apt. 13 (data not shown). Based on these binding results, Apt. 1 was selected for downstream analysis as it was also most enriched in the final round.

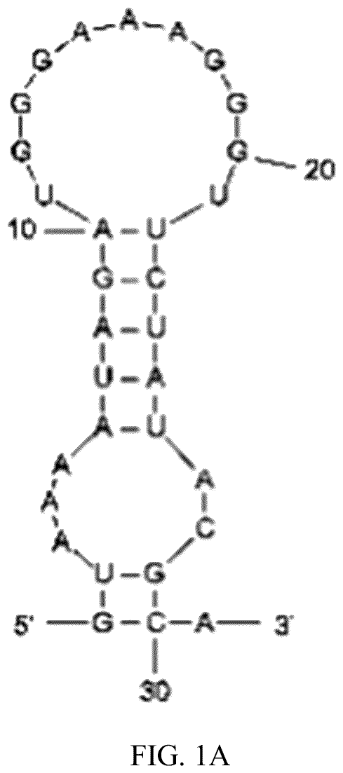

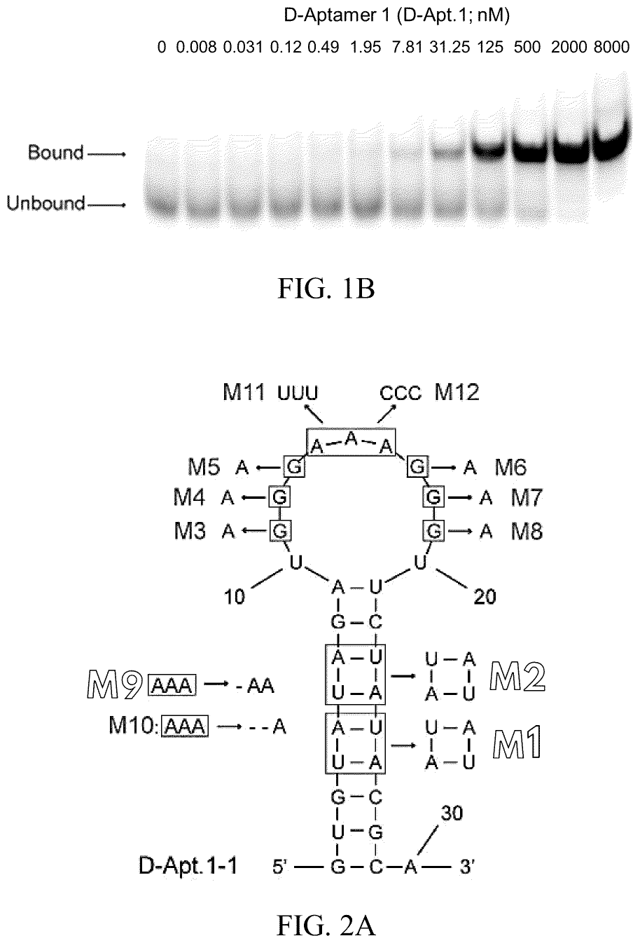

The Apt. 1 sequence of comprises the nucleotide “GUAAAAUAGAUGGGAAAGGGUUCUAUACGCA” (SEQ ID NO: 6). According to Mfold prediction, Apt. 1 possesses a hairpin loop structure and an internal loop on the stem region (FIG. 1A). An extra 5′ G was appended for in vitro RNA transcription purpose. EMSA full-range binding test was first performed for D-Apt. 1 against FAM APP L-rG4; the results indicated that with increasing concentrations of D-Apt. 1, the fluorescent signals emitted from unbound FAM APP L-rG4 target shift upwards, indicating the interaction between D-Apt. 1 and FAM APP L-rG4 and thus the slower migrating band on the gel (bound) (data not shown). The K_{d} was determined to be 66.87±7.07 nM for the T7 in vitro transcribed RNA oligonucleotide (data not shown), and was similar with the binding affinity using a chemically synthesized RNA oligonucleotide, with a K_{d} value of 42.28±4.48 nM (FIG. 1B).

To summarize, novel non-G4-containing aptamers targeting APP rG4 with low GC N30 library input and NGS readout were identified in this example, and it was found that D-Apt. 1 was able to bind APP L-rG4 target with nanomolar binding affinity.

### Example 2 Spectroscopic and Mutagenesis Analysis Reveals the Non-rG4-Containing Structure of D-Apt.1-1 and the Key Nucleotides for Target Recognition

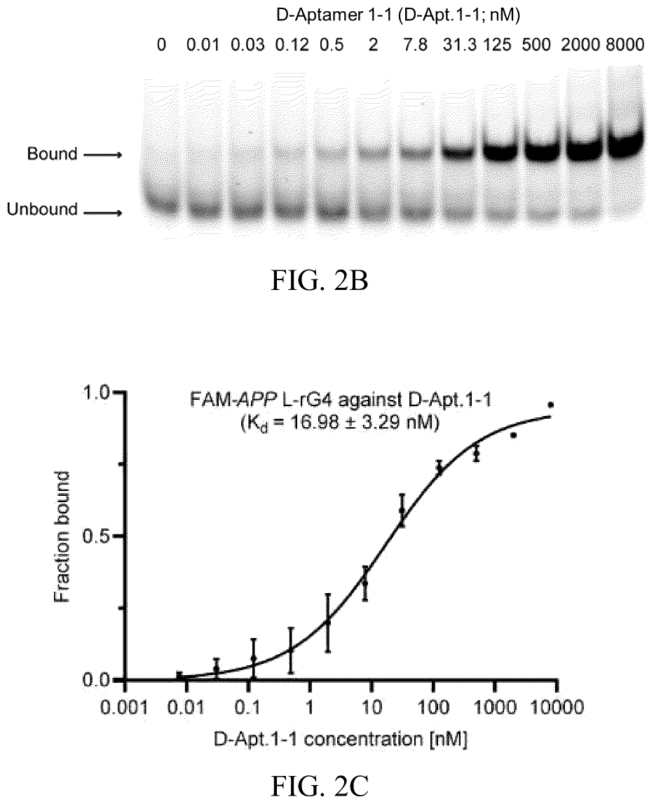

To improve the binding affinity of D-Apt. 1 to APP L-rG4 target, its stem was strengthened by replacing the internal loop with GC base pair to stabilize the hairpin loop structure, referred to as D-Apt. 1-1 that had the nucleotide sequence of “GUGUAUAGAUGGGAAAGGGUUCUAUACGCA” (SEQ ID NO: 1) as depicted in FIG. 2A. EMSA full-range binding assay was performed. According to the results, D-Apt. 1-1 had improved binding affinity to APP L-rG4, with a dissociation constant (K_{d}) of 16.98±3.29 nM (FIGS. 2B and 2C), comparing to D-Apt. 1 of 42.28±4.48 nM.

The G4 prediction result mentioned above for D-Apt. 1 and D-Apt. 1-1 suggested them to have low probability to form G4 (data not shown). Next, multiple spectroscopic analysis for G4 detection purposes was performed to see if D-Apt. 1-1 would form a G4 structure or not. Using circular dichroism, the results indicated no significant enhancement peaks at 240 nm and 260 nm in the case of K′ compared to Li′ conditions, indicating no G4 structure forming in D-Apt. 1-1 (data not shown). To further support the findings, ligand-enhanced fluorescence assay was performed using G4-specific ligands, including NMM and ThT. The results indicated the ligand fluorescence was independent of K′ and Lit conditions in the presence of D-Apt. 1-1, suggesting no rG4 formation in D-Apt. 1-1 (data not shown). These findings validated the G4 prediction result and indicated that D-Apt. 1-1 do not contain any G4 motif in the aptamer structure.

The Mfold program predicted the RNA secondary structure of D-Apt. 1-1 as a hairpin structure (FIG. 2A). We hypothesized that the conserved sequence on the loop, i.e. “UGGGAAAGGGU” (SEQ ID NO: 2), is for its binding to APP L-rG4 due to its high occurrence in NGS sequencing data, and the base pair co-variation observed in the stem region, as well as variation in stem length (data not shown), implied that the stem structure are likely important for the binding. To investigate the interaction between D-Apt.1-1 and APP L-rG4, mutagenesis analysis was performed on D-Apt. 1-1. 12 mutated constructs of D-Apt. 1-1 were designed to evaluate the effect of sequence and structure towards APP L-rG4 target binding using EMSA. Two mutants (M1 & M2) that flipped two of the base pairs in the stem region were first designed. The results indicated these co-variation mutants had similar binding to wildtype, suggesting the base pair structure rather than the sequence in the stem was important for binding, validating the base pair covariation data observed from the NGS (data not shown). Next, the Gs on the loop to As (M3-M8) were mutated to verify whether the bases on the loop were needed for binding, and the binding was disrupted in all constructs (data not shown). Then, the As on the loop were deleted, and the results indicated that deleting one A base could weaken the binding (M9), and deleting two A bases completely disrupted the binding (M10), indicated that the binding was sensitive to the number of A bases (data not shown). Lastly, the AAA on the loop to UUU (M11) and CCC (M12) were mutated, and weak binding was observed in M11 and no binding was observed in M12, illustrating that the AAA sequence was important for binding (data not shown).

Overall, the data demonstrated experimentally that D-Apt. 1-1 indeed does not contain G4 motif, and the loop region sequence is for target recognition, and the stem region hold the aptamer structure into a hairpin structure.

### Example 3 Characterization of L-Apt.1-1

### 3.1 L-Apt.1-1 Specifically Recognizes D-APP rG4 Over Non-Targets, and the Interaction is Magnesium Ion Dependent

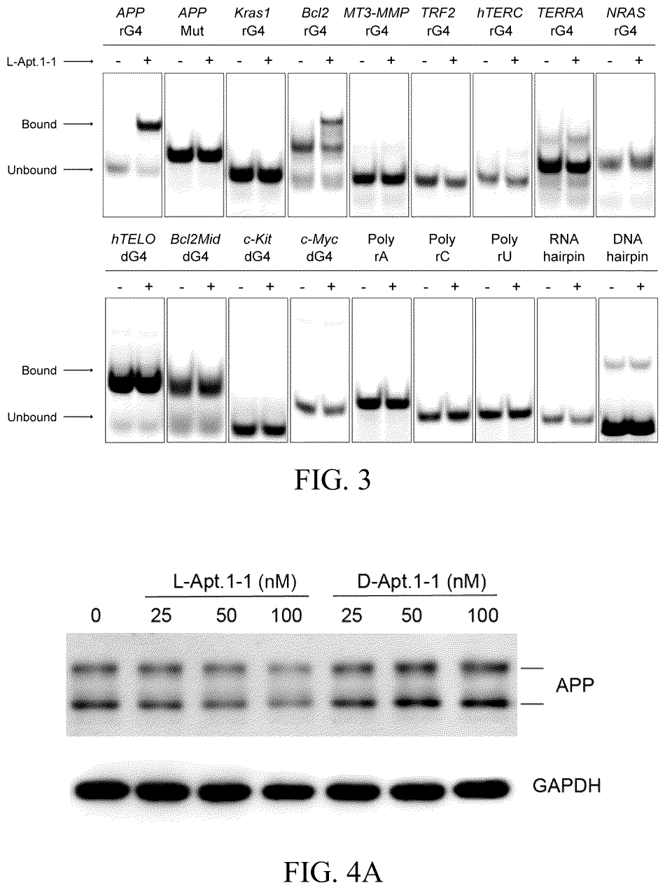

The binding specificity of L-Apt. 1-1 was investigated in this example by binding with 18 constructs via EMSA, including several rG4s (Kras1, Bcl2, MT3-MMP, TRF2, hTERC, TERRA, NRAS), dG4s (hTELO, Bcl2Mid, c-Kit, c-Myc), RNA/DNA hairpins, single-stranded RNAs (poly rA/rC/rU), APP rG4 and APP rG4 mutant. No binding was observed for all dG4s, RNA/DNA hairpins and single-stranded RNAs, indicating the high specificity of L-Apt. 1-1 (FIG. 3). Given that APP rG4 (intended target) and other rG4s may share quite similar structural features, it is generally difficult to develop L-RNA aptamer that targets a specific rG4 motif, and our previous attempt using L-Apt.8f to target APP D-rG4 was not entirely successful, as it was found to bind strongly to 2 of the unintended targets, Bcl2 rG4 and Kras1 rG4. We thus hypothesized that non-G4-containing L-Apt. 1-1 might perform better in the binding specificity test. Therefore, the binding of L-Apt. 1-1 to eight rG4 constructs was tested to evaluate its binding specificity among rG4 structures. The results demonstrated that the binding of L-Apt. 1-1 was specific to APP rG4, except it also bond weakly to one of the rG4 non-targets (Bcl2) (FIG. 3). The binding of L-Apt. 1-1 to APP D-rG4 and Bcl2 D-rG4 were further compared by performing full-range EMSA. The results indicated that L-Apt. 1-1 bound approximately 20-fold stronger to APP D-rG4 than Bcl2 D-rG4, with a dissociation constant (K_{d}) of 12.48±2.82 nM and 266.0±27.2 nM, respectively (data not shown). The analytic result of full binding for the Kras/rG4 was in agreement with FIG. 3, confirmed that no binding was detected, and the K_{d} was too weak to be determined (data not shown). Remarkably, the specificity results for L-Apt. 1-1 was in great contrast to rG4-containing L-RNA aptamer L-Apt.8f, in which it recognized not only to APP rG4 (K_{d} 13.02±1.42 nM), but also to Bcl2 rG4 and Kras1 rG4 strongly, with K_{d}s of, 39.57±8.25 nM and 71.74±17.10 nM respectively, under the same reaction conditions (data not shown). The binding specificity results demonstrated that L-Apt. 1-1 preferentially bound to the intended target APP rG4.

Next, the enantiomeric specificity of Apt. 1-1 were also tested, and the results indicated that Apt. 1-1 was enantiomeric-specific that could only bind to target in its mirror form, i.e. APP D-rG4-L-Apt. 1-1 or APP L-rG4-D-Apt.1-1, but not APP D-rG4-D-Apt.1-1 or APP L-rG4-L-Apt.1-1 (data not shown). Last, the magnesium ion dependence of the binding of L-Apt. 1-1 to APP D-rG4 under 0 mM, 1 mM or 2 mM MgCl₂ was investigated using EMSA. The K_{d}s were determined to be 54.13±5.0 nM, 12.48±2.82 nM and 3.88±1.50 nM respectively, suggesting that magnesium ions could enhance the binding between L-Apt. 1-1 and APP D-rG4.

To summarize, L-Apt. 1-1 is highly specific to APP D-rG4 over other non-targets, and the binding was magnesium ion dependent and RNA chirality dependent.

### 3.2 Application of L-Apt.1-1 to Regulate APP Expression in Cells

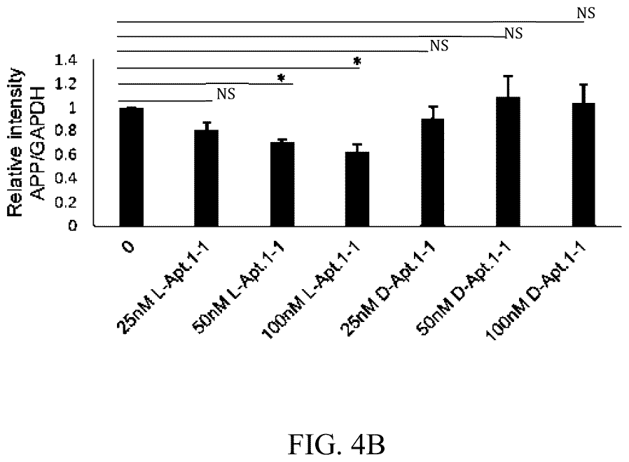

To test whether L-Apt. 1-1 can regulate endogenous APP protein expression by the interaction with the APP 3′UTR rG4 structure, cells were transfected with different concentration of L-Apt. 1-1 (0 nM, 25 nM, 50 nM and 100 nM), and the endogenous APP level was detected by western blot (data not shown). D-Apt.1-1 as negative control was also transfected, as it did not bind to the APP D-rG4 target (data not shown). The result indicated that L-Apt. 1-1 can inhibit endogenous APP expression in a concentration dependent manner. Compared with non-treated group (0 nM), the APP protein expression levels treated with different concentrations of L-Apt. 1-1 (25 nM, 50 nM and 100 nM) were reduced by approximately 19%, 30% and 38% respectively (FIGS. 4A and 4B). Consistent with the in vitro binding assay, the D-Apt. 1-1 treated group also exhibited no effect on the APP expression level (FIGS. 4A and 4B).

To further assess whether the inhibition effect is due to the transcription or translation, quantitative reverse transcription-polymerase chain reaction (qRT-PCR) was performed to detect the mRNA level of APP. The result indicated that neither L-Apt. 1-1 nor D-Apt. 1-1 affected the mRNA level of APP (data not shown), suggesting that the inhibition effect of L-Apt. 1-1 is at the translation level but not at the transcriptional level.

In addition, the stability of Apt.1-1 in biological environment was also tested with the treatment of 5% fetal bovine serum (FBS) or 100 ng/μL RNase A. Low degradation of L-Apt. 1-1 was observed in 2 hours, while D-Apt. 1-1 was all degraded within 30 minutes (data not shown), indicating the enhanced biostability of L-oligonucleotides potential for biological applications.

Taken together, L-Apt. 1-1 is biostable, and is capable of inhibiting the translation of APP via targeting APP 3′UTR rG4 motif in cells.

### Example 4 Development of the Bi-Functional FLAP Using Apt.1-1 and Pepper Aptamer Allows APP rG4 Detection In Vitro and in Cells

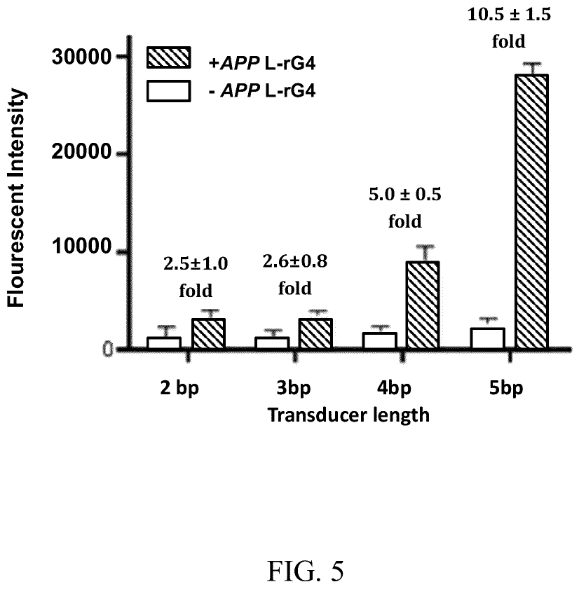

Recently, the pepper aptamer was used for the detection of small molecules and proteins. However, pepper-based rG4 detection has not been developed, and the L-pepper system was not investigated. Here, L-Apt. 1-1 was used as a pepper-based rG4 sensor to achieve specific fluorescence light-up against APP rG4, by designing a bi-functional FLAP that binds to both HBC530 ligand and APP rG4 in different motifs. We hypothesized that the HBC530 ligand could bind to L-pepper with similar affinity to D-Pepper as it was achiral. First, the system in D-form was tested, and three bi-functional FLAPs with transducer lengths ranging from one to three base pairs at the P1 stem (P1_1 bp, P1_2 bp, P1_3 bp) were designed (Table 1). The fluorescent light-up of HBC530 ligand was tested in the presence of bi-functional FLAPs with or without APP L-rG4, and the results indicated that all samples exhibited low fluorescent signal when APP L-rG4 was absent, and restored fluorescence when APP L-rG4 was present, with a best fold effect of 5.3±1.9 (data not shown). This indicated the bi-functional FLAPs could only regenerate the HBC binding pocket and enhance HBC fluorescence by binding to APP L-rG4 target that provide extra stabilization. Whether fusion of Apt. 1-1 to the P3 stem of pepper aptamer could improve the fold effect was then tested. Four bi-functional FLAPs with transducer lengths ranging from two to five base pairs (P3_2 bp, P3_3 bp, P3_4 bp, P3_5 bp) were designed (Table 1). According to the results, the performance was improved, with a best fold effect of 10.5±1.5 from P3_5 bp (D-Apt.1-1_Pepper) (FIG. 5).

TABLE 1
Nucleotide sequence of specified FLAP

| Name | Nucleotide sequence | SEQ ID NO |
|---|---|---|
| Bi-functional FLAP | GGCGCCAAUCGUAGCGUGUCGGUGGGAAAGG | 7 |
| (P1_1 bp) | GUCACUGGCGCUGCGCC | |
| Bi-functional FLAP | GGCGCCAAUCGUAGCGUGUCGGCUGGGAAAGG | 8 |
| (P1_2 bp) | GUGCACUGGCGCUGCGCC | |
| Bi-functional FLAP | GGCGCCAAUCGUAGCGUGUCGGCGUGGGAAAG | 9 |
| (P1_3 bp) | GGUCGCACUGGCGCUGCGCC | |
| Bi-functional FLAP | GGCGCACUGGCGCUGCUGGGAAAGGGUGCCAA | 10 |
| (P3_2 bp) | UCGUAGCGUGUCGGCGCC | |
| Bi-functional FLAP | GGCGCACUGGCGCUGCGUGGGAAAGGGUCGCC | 11 |
| (P3_3 bp) | AAUCGUAGCGUGUCGGCGCC | |
| Bi-functional FLAP | GGCGCACUGGCGCUGCGCUGGGAAAGGGUGCG | 12 |
| (P3_4 bp) | CCAAUCGUAGCGUGUCGGCGCC | |
| D-Apt.1-1_Pepper | GGCGCACUGGCGCUGCGCCUGGGAAAGGGUGG | 3 |
| (P3_5 bp) | CGCCAAUCGUAGCGUGUCGGCGCC | |

To further improve its fold effect, the system concentration was reduced by half, using 0.5 μM of D-Apt. 1-1_Pepper, and the fold effect was successfully improved to 14.2±1.6 fold (data not shown). Whether the concentration of HBC530 ligand affected the fluorescent signal was also investigated by reducing the pepper:HBC ratio from 1:5 to 1:2, and the fold effect was improved to 30.0±3.6 (data not shown). Then, whether the fluorescent light-up of the D-Apt. 1-1_Pepper system was dependent on the target concentrations was tested. The results indicated that the fluorescent signals were enhanced with increasing APP L-rG4 concentrations, with a highest fold of 47.8±3.7, indicating that the system could be applied to detect target in different concentrations (data not shown).

Next, the performance of L-Apt. 1-1_Pepper was investigated; the results indicated that L-Apt. 1-1_Pepper could response to different APP D-rG4 concentrations in the presence of HBC530 ligand, with the highest fold of 44.5±1.4 (data not shown). As provided in the original study on the magnesium ions dependency of the pepper-HBC interaction and the magnesium ions dependency of L-Apt.1-1-APP D-rG4 interaction, the influence of magnesium ions concentrations on L-Apt. 1-1_Pepper system was investigated. The results indicated that under 0 mM magnesium ion, no fluorescent light-up could be observed, while the fluorescent signal increased in 5 mM magnesium ions, indicating that the system was magnesium ion dependent (data not shown). Given that L-Apt. 1-1 had a high specificity to APP D-rG4 as demonstrated in Example 3.1, specificity test on L-Apt. 1-1_Pepper against APP D-rG4 and other non-targets (including DNA hairpin, dG4, rG4 and APP L-rG4) was performed. Enhanced fluorescent signal could be observed in the presence of APP D-rG4, but not in other non-targets, indicating that the L-Apt. 1-1_Pepper system can achieve specifically fluorescent light-up of its intended target (data not shown). To summarize, L-Apt. 1-1_Pepper can specifically detect APP D-rG4 in a concentration dependent manner in vitro.

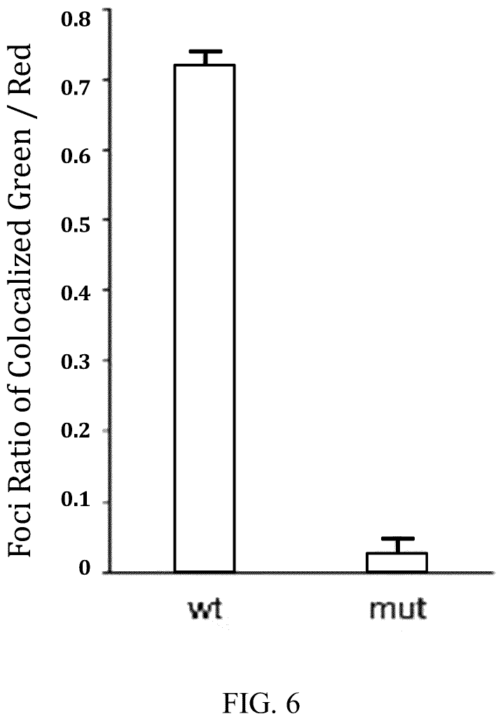

Then, cellular imaging of APP D-rG4 using L-Apt. 1-1_Pepper was performed. Due to the low abundance of endogenous mRNA, it is hard to detect rG4 directly. Thus, cells were transfected with TAMRA-labeled APP rG4 motif RNA for detection. To show the light-up fluorescence is rG4 structure dependent, a rG4 mutant group (mut) was also designed as control by the mutation of middle G of the G tracts to A to disrupt G4 structure. After transfection, cells were stained with L-Apt. 1-1_Pepper in the presence of HBC530 and scanned by confocal microscopy. According to the result, both wt and mut group showed red TAMRA foci (data not shown), indicating the successful transfection of wt/mut APP rG4 oligos into Hela cells. For wt group, L-Apt. 1-1_Pepper foci were observed, and the L-Apt. 1-1_Pepper foci colocalized well with the TAMRA signal at the ratio of about 72% (FIG. 6), indicating that L-Apt. 1-1_Pepper recognized APP rG4 in cells and was light-up upon binding to APP rG4 in the presence of HBC530. For mut group, however, there was almost no L-Apt. 1-1_Pepper signal at all, with the co-localization ratio of about 3% (FIG. 6), indicating the recognition and light-up mode was rG4 structure dependent. Besides, L-Apt. 1-1_Pepper was applied in another cell line, HEK293T cell. The confocal imaging depicted a similar result that L-Apt. 1-1_Pepper can be light-up well only in wt group (about 67% co-localization ratio), while not in mut group (about 2% co-localization ratio) (data not shown), further confirming the general use of the probe.

Overall, L-Apt. 1-1_Pepper can be used to visualize the specific APP rG4 structure in cells as a light-up probe.

It will be understood that the above description of embodiments is given by way of example only and that various modifications may be made by those with ordinary skill in the art. The above specification provides a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. An L-form ribonucleic acid (L-RNA) aptamer specific to the 3′-untranslated region (3′-UTR) of amyloid precursor protein (APP) messenger RNA (mRNA), comprising the nucleotide sequence of SEQ ID NO: 1, wherein the L-RNA aptamer is characterized by having a loop region consisting of the nucleotide sequence of SEQ ID NO: 2.

2. The L-RNA aptamer of claim 1, wherein the L-RNA aptamer is produced by a method comprising the steps of,
(a) providing a deoxyribonucleic acid (DNA) library comprising a first plurality of DNAs respectively having randomized sequences;
(b) producing a first plurality of D-form RNAs (D-RNAs) respectively corresponding to the first plurality of DNAs of step (a) via in vitro transcription;
(c) mixing the first plurality of D-RNAs of step (b) with a first plurality of streptavidin-coated magnetic beads, which are pre-treated with transfer RNAs (tRNAs);
(d) collecting and mixing the supernatant of the product of step (c) with a biotinylated L-form nucleic acid having the nucleotide sequence of SEQ ID NO: 4;
(e) mixing the product of step (d) with a second plurality of streptavidin-coated magnetic beads, which are pre-treated with the tRNAs;
(f) eluting the precipitate of step (e) with an elution buffer so as to produce a second plurality of D-RNAs specific to the L-form nucleic acid;
(g) producing a plurality of complementary DNAs (cDNAs) respectively corresponding to the second plurality of D-RNAs of step (f) via reverse transcription;
(h) producing a second plurality of DNAs respectively corresponding to the plurality of cDNAs of step (g) via polymerase chain reaction (PCR);
(i) repeating steps (b) to (h) at least 4 times, in which the in vitro transcription was performed by using the product of step (h), so as to identify a D-RNA aptamer specific to the L-form nucleic acid; and
(j) producing the L-RNA aptamer corresponding to the D-RNA aptamer of step (i).

3. The L-RNA aptamer of claim 2, wherein the ratio of the sum of the adenine (A) and thymine (T) nucleotides to the sum of the cytosine (C) and guanine (G) nucleotides in each randomized sequence is about 7:3.

4. The L-RNA aptamer of claim 2, wherein the method further comprises,
(c-1) subjecting the product of step (c) to centrifugation or magnetic field prior to step (d).

5. The L-RNA aptamer of claim 2, wherein the method further comprises,
(e-1) subjecting the product of step (e) to centrifugation or magnetic field prior to step (f).

6. A recombinant polynucleotide having the loop region of the L-RNA aptamer according to claim 1, comprising the nucleotide sequence of SEQ ID NO: 3.

7. A method of treating Alzheimer's disease (AD) in a subject comprising administering to the subject an effective amount of the L-RNA aptamer of claim 1 to alleviate or ameliorate the symptoms associated with the AD.

8. The method of claim 7, wherein the subject is a human.
